(19) United States
(12) Patent Application Publication
(10) Pub. No.: US 2016/0081722 A1
(43) Pub. Date: Mar. 24, 2016

(54) SURGICAL IMPLANT SYSTEM AND METHOD

(71) Applicant: Warsaw Orthopedic, Inc., Warsaw, IN (US)

(72) Inventors: Loic Josse, Yens Vaud (CH); Jerome Nayet, St. Genis Pouilly (FR)

(21) Appl. No.: 14/490,351

(22) Filed: Sep. 18, 2014

(51) Int. Cl. A61B 17/70; A61B 17/80

(52) U.S. Cl. CPC A61B 17/7059; A61B 17/8052

(57) ABSTRACT

A spinal implant includes a wall that defines at least one opening and includes at least one part extending within the opening for disposal with a groove surface of a fastener. The fastener defines an axis. The groove surface is disposed between a head and a tissue penetrating shaft of the fastener. The at least one part is engageable with the groove surface to resist and/or prevent axial movement of the fastener relative to the wall. Systems and methods are disclosed.

### TECHNICAL FIELD

The present disclosure generally relates to medical devices for the treatment of musculoskeletal disorders, and more particularly to a surgical system and a method for treating a spine.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. As part of these surgical treatments, implants such as bone fasteners, plates, connectors and vertebral rods are often used to provide stability to a treated region. These implants can redirect stresses away from a damaged or defective region while healing takes place to restore proper alignment and generally support the vertebral members. For example, plates may be attached via the fasteners to the exterior of one or more vertebral members. This disclosure describes an improvement over these prior art technologies.

### SUMMARY

In one embodiment, a spinal implant is provided. The spinal implant includes a wall that defines at least one opening and includes at least one part extending within the opening for disposal with a groove surface of a fastener. The fastener defines an axis. The groove surface is disposed between a head and a tissue penetrating shaft of the fastener. The at least one part is engageable with the groove surface to resist and/or prevent axial movement of the fastener relative to the wall. In some embodiments, systems and methods are provided.

### BRIEF DESCRIPTION OF THE DRAWINGS

The present disclosure will become more readily apparent from the specific description accompanied by the following drawings, in which:

FIG. 1 is a perspective view of components of one embodiment of a surgical system in accordance with the principles of the present disclosure;

FIG. 2 is a perspective view of the components shown in FIG. 1;

FIG. 3 is an enlarged view of detail A shown in FIG. 1;

FIG. 4 is a perspective view of components of one embodiment of a surgical system in accordance with the principles of the present disclosure;

FIG. 5 is a side view of components of one embodiment of a surgical system in accordance with the principles of the present disclosure;

FIG. 6 is a side view of components of one embodiment of a surgical system in accordance with the principles of the present disclosure;

FIG. 7 is a perspective view of components of one embodiment of a surgical system in accordance with the principles of the present disclosure;

FIG. 8 is a perspective view of components of one embodiment of a surgical system in accordance with the principles of the present disclosure;

FIG. 9 is a cross section view of components of one embodiment of a surgical system in accordance with the principles of the present disclosure;

FIG. 10 is an enlarged view of detail B shown in FIG. 7;

FIG. 11 is a perspective view of components of one embodiment of a surgical system in accordance with the principles of the present disclosure;

FIG. 12 is a side view of components of one embodiment of a surgical system in accordance with the principles of the present disclosure; and

FIG. 13 is a perspective view, in part cross section, of components of one embodiment of a surgical system in accordance with the principles of the present disclosure.

### DETAILED DESCRIPTION

The exemplary embodiments of the surgical system and related methods of use disclosed are discussed in terms of medical devices for the treatment of musculoskeletal disorders and more particularly, in terms of a spinal implant system and a method for treating a spine.

In one embodiment, the present system includes an implant, such as, for example, an anterior cervical plate including an anti-back out mechanism that is monolithically formed and/or integrally connected with the plate. In some embodiments, the anti-back out mechanism can include a part of a plate design and does not require sub-components to be manufactured and assembled. In some embodiments, upon finalizing the insertion of bone screws in plate holes, the screw heads are automatically secured without an additional step. This configuration provides reliability that the implanted screws are secured.

In some embodiments, the plate provides retention and/or locking of screws with the plate with elastic tongues on the plate that are disposable in a groove below a screw head engageable with the plate. In some embodiments, the retention and/or locking is below the plate surface and will not impinge soft tissue of the esophagus. In some embodiments, this configuration decreases irritation. In some embodiments, this configuration facilitates surgery and includes self-locking of screws with the plate such that a step of a surgeon locking the screws is not required.

In some embodiments, the plate includes a circular undercut creating one flexible part in each screw hole of the plate. In some embodiments, the flexible part is slanted to facilitate screw insertion and oppose back out. In some embodiments, the flexible part fits in the bone screws where a specific groove is designed to receive the flexible part such that each screw is fully inserted in the plate and automatically secured. In some embodiments, the anti-back out mechanism is reversible as the groove in the screw has multiple ramps allowing the flexible part to engage the screw in a reverse orientation. In some embodiments, the removal torque of the screw is determined by the stiffness of the flexible part and avoids screw migration due to vibration only. In some embodiments, the flexible parts are selectively positioned to allow a cephalad-caudal motion of an angle of 25 degrees. In some embodiments, the flexible parts are selectively positioned to allow a cephalad-caudal motion of an angle of 17 degrees.

In some embodiments, the plate includes a circular undercut creating two flexible parts in the plate. In some embodiments, the flexible parts are slanted to facilitate screw insertion and oppose the back out. In some embodiments, the flexible parts fit in the bone screws such that a specific groove is designed to receive the flexible parts such that each screw is fully inserted in the plate and automatically permanently secured. In some embodiments, the anti-back out mechanism is non-reversible.

In some embodiments, the plate includes two cuts creating a flexible part in the plate. In some embodiments, the flexible part has a shape of a hammer. In some embodiments, a hammer head fits in the bone screws such that a groove receives the hammer head. In some embodiments, once the second screw is inserted in the plate, the screws are permanently secured. In some embodiments, the hammer ends are selectively positioned to allow a cephalad-caudal motion of an angle of 25 degrees. In some embodiments, the hammer ends are selectively positioned to allow a cephalad-caudal motion of an angle of 17 degrees.

In some embodiments, the plate includes a circular undercut creating multiple flexible parts in the plate. In some embodiments, the flexible parts are slanted to facilitate screw insertion and oppose back out. In some embodiments, the flexible parts fit in the bone screws where a selected groove receives the flexible parts such that each screw is fully inserted in the plate and automatically permanently secured. In some embodiments, the flexible parts are positioned to allow rotation in all directions.

In some embodiments, the plate is employed with a bone screw that includes a groove having a V-shaped cross-section configuration. In some embodiments, the V-shaped cross-section allows adjustment of the removal force or torque. In some embodiments, the bone screw includes one or a plurality of flats, such as, for example, two or three flats that are selectively placed to align with one or two blades of the plate to force the opening of blade(s).

In one embodiment, one or all of the components of the surgical system are disposable, peel-pack, pre-packed sterile devices that can be used with an implant. One or all of the components of the surgical system may be reusable. The surgical system may be configured as a kit with multiple sized and configured components.

In some embodiments, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed surgical system and methods may be alternatively employed in a surgical treatment with a patient in a prone or supine position, and/or employ various surgical approaches to the spine, including anterior, posterior, posterior mid-line, direct lateral, postero-lateral, and/or antero-lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, in some embodiments, as used in the specification and including the appended claims, the singular forms “a,” “an,” and “the” include the plural, and reference to a particular numerical value includes at least that particular value, unless the context dearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

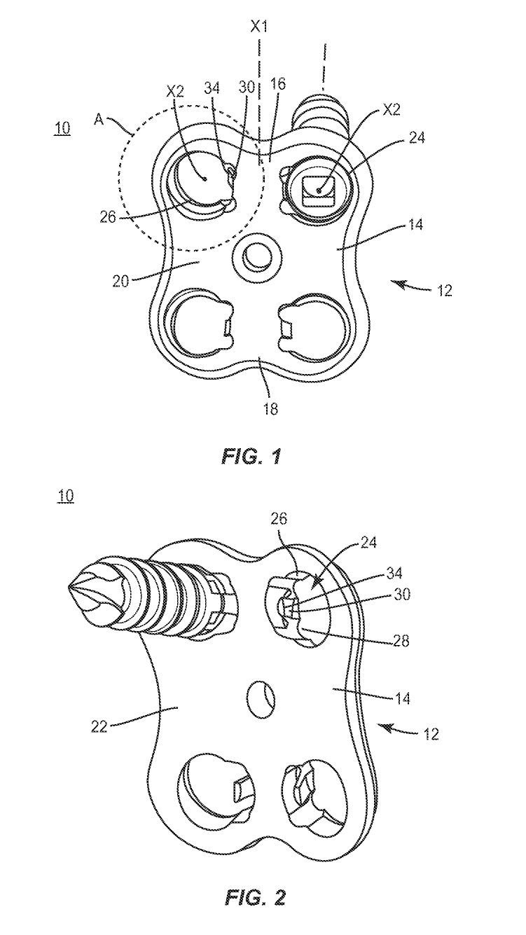

The following discussion includes a description of a surgical system and related methods of employing the surgical system in accordance with the principles of the present disclosure. Alternate embodiments are also disclosed. Reference is made in detail to the exemplary embodiments of the present disclosure, which are illustrated in the accompanying figures. Turning to FIGS. 1-4, there are illustrated components of a surgical system 10, including a spinal implant 12 in accordance with the principles of the present disclosure.

The components of surgical system 10 can be fabricated from biologically acceptable materials suitable for medical applications, including metals, synthetic polymers, ceramics and bone material and/or their composites. For example, the components of surgical system 10, individually or collectively, can be fabricated from materials such as stainless steel alloys, commercially pure titanium, titanium alloys, Grade 1, 2, 3, 4 or 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of surgical system 10 may have material composites, including the above materials, to achieve various desired characteristics such as strength, rigidity, elasticity, compliance, biomechanical performance, durability and radiolucency or imaging preference. The components of surgical system 10, individually or collectively, may also be fabricated from a heterogeneous material such as a combination of two or more of the above-described materials. The components of surgical system 10 may be monolithically formed, integrally connected or include fastening elements and/or instruments, as described herein.

The components of surgical system 10 including spinal implant 12 can be employed, for example, with a minimally invasive procedure, including percutaneous techniques, mini-open and open surgical techniques to deliver and introduce an implant, such as, for example, one or a plurality of bone fasteners and/or spinal plates, at a surgical site within a body of a patient, for example, a section of a spine.

Spinal implant 12 comprises, such as, for example, an anterior cervical plate. Plate 12 has a substantially rectangular configuration and defines a longitudinal axis X1. In some embodiments, plate 12 can be variously configured, such as, for example, tubular, oval, oblong, triangular, square, polygonal, irregular, uniform, non-uniform, variable, hollow and/or tapered. In some embodiments, plate 12 may have a curvature, angled portions and/or geometry relative to and/or along axis X1 that mates with an anatomical curvature and/or other geometry of a spine. Plate 12 includes a wall 14 extending between an end 16 and an end 18. Wall 14 has a substantially rectangular cross section and defines a thickness. In some embodiments, wall 14 can have alternate cross-section and/or thickness configurations, such as, arcuate, undulating, offset, staggered, tubular, oval, oblong, triangular, square, polygonal, irregular, uniform, variable, hollow and/or tapered.

Plate 12 includes a surface 20 and a surface 22. Surface 22 includes substantially planar portions and is oriented in a first direction such that all or only a portion of surface 22 faces and/or engages tissue, as described herein. Surface 20 is oriented in a second direction, opposite to the first direction. In one embodiment, plate 12 is configured for engagement with an anterior portion of vertebral tissue. In one embodiment, surface 22 has a frictional surface configuration for engagement with tissue to enhance fixation. In some embodiments, surface 22 may include alternate surface configurations, such as, for example, rough, arcuate, undulating, mesh, porous, semi-porous, dimpled and/or textured.

Plate 12 has a wall 14 includes surface 15 that defines openings 24. Each opening 24 extends between surfaces 20, 22 and defines a longitudinal axis X2 disposed transverse to axis X1, as shown in FIG. 3. Openings 24 are substantially circular and extend through the thickness of wall 14. In some embodiments, axis X2 may be disposed at alternate orientations, relative to axis X1, such as, for example, substantially transverse, perpendicular and/or other angular orientations such as acute or obtuse, and/or may be offset. In some embodiments, openings 24 can be variously configured, such as, for example, oval, oblong, triangular, square, polygonal, irregular, uniform, non-uniform and/or tapered. In some embodiments, plate 12 can have two or more openings 24.

Surface 15 includes a flange 26. Range 26 extends circumferentially about each opening 24 between an end 26a and an end 26b. Range 26 is configured for engagement with a neck of a bone fastener, such as, for example, when a bone screw is seated with plate 12. End 26a and end 26b define a cavity 28.

Surface 15 defines a part, such as, for example, a projection 30. Projection 30 is flexible and extends in a resilient configuration from surface 15. Projection 30 is disposed with cavity 28 and extends into opening 24. Projection 30 includes an engagement surface, such as, for example, a tip 34 configured to engage a neck of a bone screw, as described herein. Tip 34 is disposed below surface 20 and adjacent surface 22 and/or a posterior surface of plate 12. In some embodiments, tip 34 and/or projection 30 are disposed adjacent surface 22 and in even, level and/or flush alignment with a posterior surface of plate 12. In some embodiments, projection 30 is disposed at an angular orientation relative to axis X2. In some embodiments, projection 30 is angled at 25 degrees relative to axis X2 to minimize insertion load and increase extraction load. In some embodiments, opening 24 orients a bone fastener to allow cephalad-caudal motion of the bone fastener. In some embodiments, projection 30 is angled relative to surface 15 of opening 24 to facilitate translation of a bone screw, as described herein. In some embodiments, tip 34 is tangent to a groove 62, as described herein, to allow angular motion and maintain tangent contact between tip 34 and the surface of groove 62.

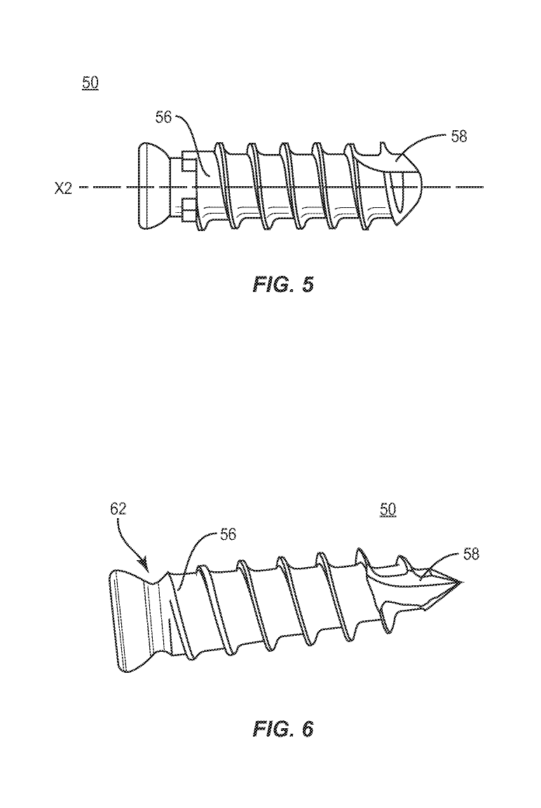

In one embodiment, opening 24 is configured to receive a bone screw 50, as shown in FIG. 4. Bone screw 50 comprises an elongated shaft 52 configured for penetrating tissue, a neck 60 and a head 54. Shaft 52 extends between an end 56 and an end 58. Shaft 52 has a cylindrical and/or conical cross section configuration and includes an outer surface having an external thread form. In some embodiments, the external thread form may include a single thread turn or a plurality of discrete threads. In some embodiments, other engaging structures may be located on shaft 52 of bone screw 50, such as, for example, a nail configuration, barbs, expanding elements, raised elements and/or spikes to facilitate engagement of shaft 52 of bone screw 50 with tissue, such as, for example, vertebrae.

Neck 60 includes a surface disposed between shaft 52 and head 54. The surface of neck 60 defines a groove 62, which is configured for disposal of tip 34. Groove 62 includes limits, such as, for example, an end 60a and an end 60b that define the bounds of groove 62 between shaft 52 and head 54. In one embodiment, groove 62 extends circumferentially about bone screw 50. In one embodiment, groove 62 extends about a portion of bone screw 60.

The surface of neck 60 includes a ramp 64 having an intermediate, raised portion 66. Ramp 64 is disposed with groove 62 and includes an incline surface that extends outwardly from the surface of neck 60 to portion 66. Ramp 64 includes a decline surface that extends from portion 66 to the surface of neck 60. In some embodiments, portion 66 may include a flat, plateau and/or arcuate surface.

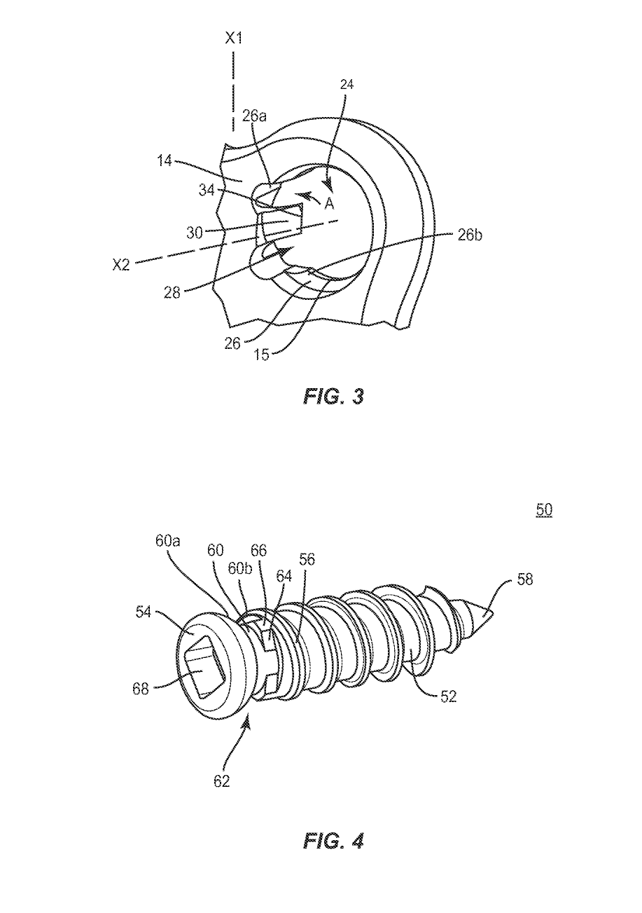

Tip 34 is engageable with ramp 64 such that the surfaces of ramp 64 circumferentially move along tip 34. As such, ramp 64 is disposed below surface 20, between surfaces 20, 22 of wall 14. In some embodiments, ramp 64 is disposed adjacent surface 22 and/or a posterior surface of plate 12. As the surfaces of ramp 64 move along tip 34, ramp 64 and portion 66 drive tip 34 downwardly to overcome the resilient bias of projection 30 such that projection 30 is deflected, for example as shown by arrow A in FIG. 3, and moved to a non-locked orientation with bone screw 50, as described herein. In some embodiments, engagement of ramp 64 with projection 30 facilitates translation of bone screw 50 relative to wall 14 for adjustment of bone screw 50. In some embodiments, neck 60 includes one or a plurality of ramps 64. In some embodiments, ramps 64 are spaced about neck 60. In some embodiments, as tip 34 translates over portion 66, tip 34 is deflected into a non-locked orientation allowing adjustment, for example in rotation and/or translation, of bone screw 50 along axis X2. In some embodiments, tip 34 moves along an inclined surface of ramp 64 such that tip 34 is gradually driven outwardly due to a taper of ramp 64 to a non-locked orientation with portion 66 and biased gradually inwardly along a decline surface of ramp 64 to a locked orientation, as shown in FIG. 2.

In one embodiment, end 58 of bone screw 50 is blunt, as shown in FIG. 5, to avoid damaging tissue and/or nerves. As shown in FIG. 5, screw 50 has a cylindrical blunt nose configuration and comprises a rescue screw, which can be employed such that in cases of screw removal, the larger end 58 and the larger diameter thereabout optimizes bone purchase left intact by a previous tapered screw. In one embodiment, groove 62 includes a V-shape, as shown in FIG. 6, configured to adjust a removal force or torque for a bone fastener via engagement with a projection 30. The V-shape of groove 62 facilitates moving projection 30, similar to ramp 64, for example, upon removal of a bone fastener. In some embodiments, a V-shape groove 62 having a sharp angle makes it difficult to remove a bone fastener and a V-shape groove 62 having a flat angle makes it easier to remove a bone fastener.

In some embodiments, all or only a portion of shaft 52 may have alternate cross section configurations, such as, for example, oval, oblong, triangular, square, polygonal, irregular, uniform, non-uniform, offset, staggered, undulating, arcuate, variable and/or tapered. In some embodiments, the outer surface of shaft 52 may include one or a plurality of openings. In some embodiments, all or only a portion of the outer surface of shaft 52 may have alternate surface configurations to enhance fixation with tissue such as, for example, rough, arcuate, undulating, mesh, porous, semi-porous, dimpled and/or textured. In some embodiments, all or only a portion of shaft 52 may be disposed at alternate orientations, relative to its longitudinal axis, such as, for example, transverse, perpendicular and/or other angular orientations such as acute or obtuse, co-axial and/or may be offset or staggered. In some embodiments, all or only a portion of shaft 52 may be cannulated.

Head 54 includes an inner surface that defines a socket cavity 68 configured for engagement with a tool or instrument for inserting and tensioning bone screw 50 with tissue and/or plate 12. Cavity 68 receives a surface of a drive element of the tool that matingly engages the inner surface of head 54 for manipulating bone screw 50. In some embodiments, the surfaces of cavity 68 and the drive element can be alternatively configured, such as, for example, thread form, triangular, square, polygonal, hexalobular, star, torx, irregular, uniform, non-uniform, offset, staggered and/or tapered.

Bone screw 50 is engageable with plate 12 to fix plate 12 with tissue. Bone screw 50 is disposed within opening 24 such that shaft 52 is translated along wall 14 and relative to axis X2 as the threads of shaft 52 rotate relative to flange 26 and projection 30. The threads of shaft 52 travel along tip 34. As shaft 52 penetrates tissue relative to wall 14, the surfaces of shaft 52 move along tip 34.

Bone screw 50 is axially translated relative to wall 14 and axis X2 until tip 34 is disposed with groove 62. As such, tip 34 is engaged with the surface of neck 60 to resist and/or prevent axial movement of bone screw 50 relative to wall 14. In some embodiments, tip 34 translates from the threads of shaft 52 directly into groove 62. In some embodiments, tip 34 engages ramps 64, as described herein, and is disposable with groove 62 therefrom. During axial translation and/or rotation of bone screw 50, tip 34 is moveable and/or flexibly configured, as shown by arrow A in FIG. 3, for positioning between a locked orientation, as shown in FIG. 2, such that tip 34 is disposed between ends 60a, 60b to resist and/or prevent axial movement of bone screw 50 relative to wall 14 and resist and/or prevent back out of bone screw 50 from tissue and a non-locked orientation such that tip 34 is movable during engagement with ramp 64, as described herein.

In the locked orientation, projection 30 is disposed in its naturally biased configuration such that tip 34 is disposed in groove 62 for engagement with ends 60a, 60b. In some embodiments, tip 34 moves along an incline of ramp 64 such that tip 34 is gradually driven downwardly due to a taper of ramp 64 to a non-locked orientation with portion 66 and biased gradually upwardly along a decline of ramp 64 to a locked orientation.

In some embodiments, all or only a portion of projection 30 may have a semi-rigid, rigid or elastic configuration, and/or have elastic properties, such as the elastic properties corresponding to the material examples described above, such that projection 30 provides a selective amount of expansion, contraction, collapse and/or extension.

In assembly, operation and use, surgical system 10 is employed to treat a selected section of vertebrae. A medical practitioner obtains access to a surgical site including the vertebrae in any appropriate manner, such as through incision and retraction of tissues. In some embodiments, surgical system 10 can be used in any existing surgical method or technique including open surgery, mini-open surgery, minimally invasive surgery and percutaneous surgical implantation, whereby the vertebrae is accessed through a mini-incision, or sleeve that provides a protected passageway to the area. Once access to the surgical site is obtained, the particular surgical procedure can be performed for treating the spine disorder.

An incision is made in the body of a patient and a cutting instrument (not shown) creates a surgical pathway for implantation of components of surgical system 10 with an anterior portion of the vertebrae. A preparation instrument (not shown) can be employed to prepare tissue surfaces of the vertebrae, as well as for aspiration and irrigation of a surgical region.

Surgical system 10 includes a surgical instrument, such as, for example, a drill, tap and screw guide (not shown). The guide is connected with plate 12 and/or bone screws 50 for orientation and delivery of the components of surgical system 10 along the surgical pathway. The guide introduces the components of surgical system 10 along the surgical pathway to implant plate 12 and/or bone screws 50 in substantial alignment to attach plate 12 and/or bone screws 50 to the vertebrae.

With plate 12 disposed in a selected orientation relative to one or more selected vertebra at a surgical site, projection 30 protrudes within opening 24. For each bone screw 50, a surgical tool (not shown) is engaged with socket cavity 68 to axially translate bone screw 50 through opening 24. Bone screw 50 is engageable with plate 12 to fix plate 12 with one or more selected vertebra at the surgical site. Bone screw 50 is disposed within opening 24 such that shaft 52 is translated along wall 14 and relative to axis X2 as the threads of shaft 52 rotate relative to flange 26 and projection 30. The threads of shaft 52 travel along tip 34. As shaft 52 penetrates tissue relative to wall 14, the surfaces of shaft 52 move along tip 34.

Bone screw 50 is axially translated relative to wall 14 and axis X2 until tip 34 is disposed with groove 62, as described herein. As such, tip 34 is engaged with the surface of neck 60 to resist and/or prevent axial movement of bone screw 50 relative to wall 14. During axial translation and/or rotation of bone screw 50, tip 34 is moveable and/or flexibly configured, as shown by arrow A in FIG. 3, for positioning between a locked orientation, as shown in FIG. 2, such that tip 34 is disposed between ends 60a, 60b to resist and/or prevent axial movement of bone screw 50 relative to wall 14, and resist and/or prevent back out of bone screw 50 from tissue, and a non-locked orientation such that tip 34 is movable during engagement with ramp 64, as described herein.

The external thread form of bone screw 50 translates along tip 34 from end 58 to end 56. Tip 34 deflects over end 60b for disposal in groove 62. Upon seating of bone screw 50 into a seated position with flange 26, tip 34 is biased into the locked orientation within groove 62 such that tip 34 is disposed within groove 62 between ends 60b, 60a. In the locked orientation, projection 30 is disposed in its naturally biased configuration, as described herein. Tip 34 resists and/or prevents axial translation of bone screw 50 relative to plate 12.

Upon completion of a procedure, as described herein, the surgical instruments, assemblies and non-implanted components of system 10 are removed and the incision(s) are closed. One or more of the components of system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of system 10. In some embodiments, system 10 may include one or a plurality of rods, plates, connectors and/or bone fasteners for use with a single vertebral level or a plurality of vertebral levels. In some embodiments, one or more of fasteners 50 may be engaged with tissue in various orientations, such as, for example, series, parallel, offset, staggered and/or alternate vertebral levels.

In one embodiment, system 10 includes an agent, which may be disposed, packed, coated or layered within, on or about the components and/or surfaces of system 10. In some embodiments, the agent may include bone growth promoting material, such as, for example, bone graft to enhance fixation of the components and/or surfaces of system 10 with vertebrae. In some embodiments, the agent may include one or a plurality of therapeutic agents and/or pharmacological agents for release, including sustained release, to treat, for example, pain, inflammation and degeneration.

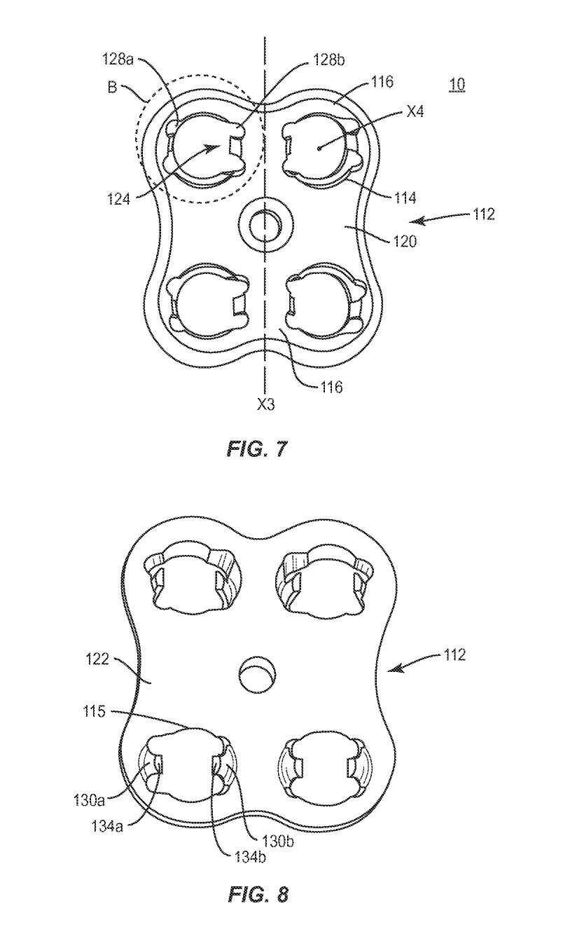

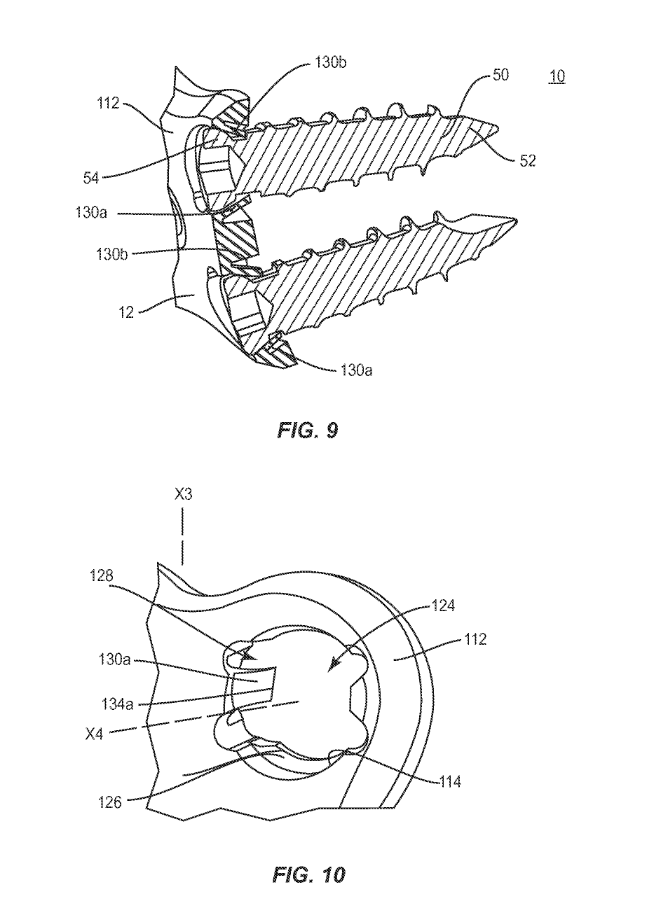

In one embodiment, as shown in FIGS. 7-10, a system 110 includes a plate 112 and one or more bone screws 50, as described herein. Plate 112 defines a longitudinal axis X3. Plate 112 includes a wall 114 extending between an end 116 and an end 118. Plate 112 includes a surface 120 and a surface 122.

Wall 114 includes surface 115 that defines openings 124. Each opening 124 extends between surfaces 120, 122 and defines a longitudinal axis X4 disposed transverse to axis X3, as shown in FIG. 10. In some embodiments, axis X4 may be disposed at alternate orientations, relative to axis X3, such as, for example, substantially transverse, perpendicular and/or other angular orientations such as acute or obtuse, and/or may be offset. Surface 115 includes a flange 126. Flange 126 extends circumferentially about each opening 124 to define cavities 128a, 128b.

Surface 115 defines projections 130a, 130b. Projections 130a, 130b are disposed with cavities 128a, 128b and extend into opening 124. Projections 130a, 130b include tips 134a, 134b configured to engage bone screw 50. Tip 134 is disposed at an orientation relative to axis X4.

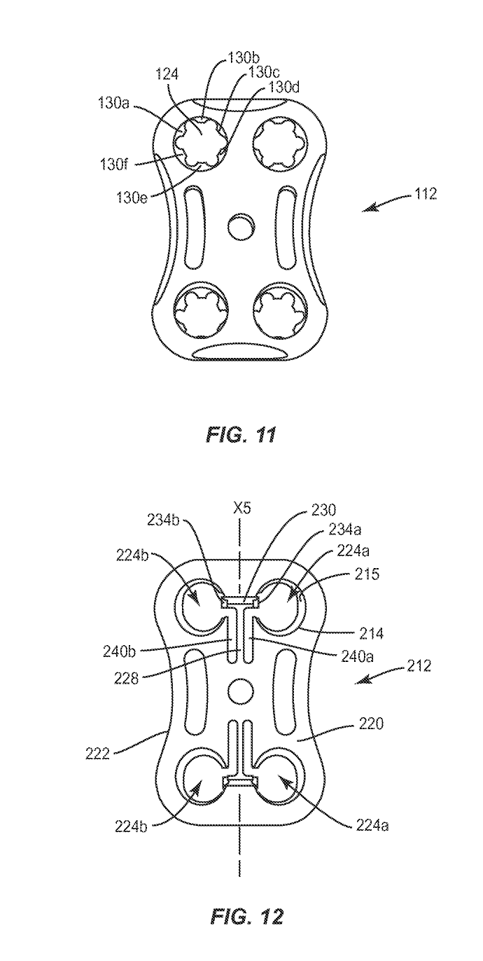

Bone screw 50 is engageable with plate 112 to fix plate 112 with tissue. Bone screw 50 is disposed with opening 124 such that shaft 52 is translated along wall 114 as the threads of shaft 52 rotate relative to flange 126 and projections 130a, 130b. Bone screw 50 is axially translated until tips 134a, 134b are disposed with groove 62 such that tips 134a, 134b are engaged with the surface of neck 60 to resist and/or prevent axial movement of bone screw 50 relative to wall 114. During axial translation and/or rotation of bone screw 50, tips 134a, 134b are moveable and/or flexibly configured for positioning between a locked orientation such that tips 134a, 134b are disposed between ends 60a, 60b to resist and/or prevent axial movement of bone screw 50 relative to wall 114 and resist and/or prevent back out of bone screw 50 from tissue and a non-locked orientation, such that tips 134a, 134b are moved during engagement with ramp 64 to facilitate adjustment of bone screw 50. In one embodiment, as shown in FIG. 11, plate 112 includes a plurality of projections 130a-130f circumferentially disposed about each opening 124.

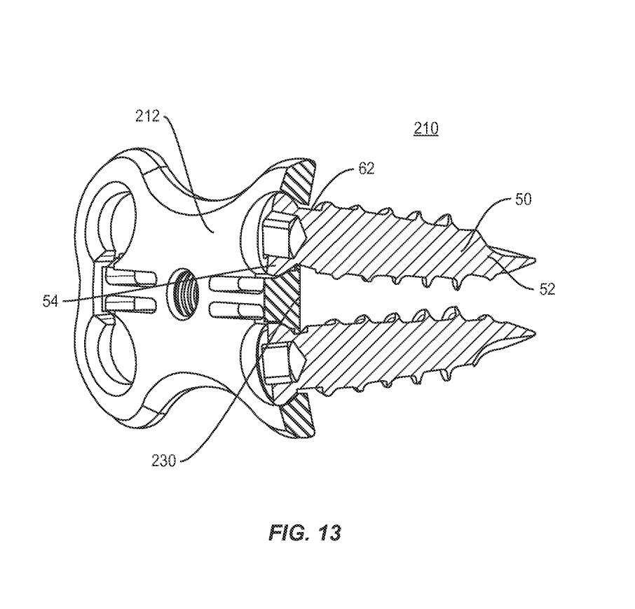

In one embodiment, as shown in FIGS. 12 and 13, a system 210 includes a plate 212 and one or more bone screws 50, as described above. Plate 212 includes a wall 214 and defines a longitudinal axis X5. Plate 212 has a surface 220 and a surface 222. Wall 214 includes a surface 215 that defines openings, such as, for example, openings 224a, 224b extending between surface 220 and surface 222.

Surface 215 defines a flexible extension 228 and a movable part 230 extending therefrom in a hammer shaped configuration. Wall 214 defines a cavity 240a and a cavity 240b configured to facilitate movement and/or rotation of extension 228 and part 230 relative to wall 214. Part 230 is configured to engage bone screw 50. Part 230 is flexible and extends in a resilient configuration from surface 215. Part 230 extends from surface 215 and includes ends 234a, 234b that extend into openings 224a, 224b.

Bone screws 50 are engageable with plate 212 to fix plate 212 with tissue. Bone screws 50 are disposed with openings 224a, 224b such that shaft 52 is translated along wall 214 as the threads of shaft 52 rotate relative to ends 234a, 234b. As shaft 52 penetrates tissue relative to wall 214, the surfaces of shaft 52 move along ends 234a, 234b. Bone screw 50 is axially translated until ends 234a, 234b are disposed with groove 62 such that ends 234a, 234b are engaged with the surface of neck 60 to resist and/or prevent axial movement of bone screw 50 relative to wall 214. During axial translation and/or rotation of bone screw 50, ends 234a, 234b are pivotally moveable relative to wall 214 and/or flexibly configured for positioning to a locked orientation such that ends 234a, 234b are disposed between ends 60a, 60b to resist and/or prevent axial movement of bone screw 50 relative to wall 214.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

1. A spinal implant comprising:
a fastener; and
a wall comprising opposite to and bottom surfaces, the wall comprising an inner surface defining at least one opening that extends through the surfaces, the wall including at least one part extending within the opening, the fastener including a groove surface and a ramp,
the at least one part comprising a tip configured for positioning between a locked orientation in which the tip engages the groove surface to resist and/or prevent axial movement of the fastener relative to the wall and a non-locked position in which the tip is movable during engagement with the ramp, the tip being positioned below the bottom surface in the locked and non-locked orientations.

2. A spinal implant as recited in claim 1, wherein the at least one part extends from the wall in a resilient configuration.

3. A spinal implant as recited in claim 2, wherein the wall includes a circumferential flange disposed within the opening and defines the at least one part.

4. A spinal implant as recited in claim 1, wherein the opening defines a longitudinal axis, the at least one part comprising a projection that is disposed at an angle relative to the longitudinal axis, the tip being a tip of the projection.

5. A spinal implant as recited in claim 4, wherein the angle is 25 degrees.

6. A spinal implant as recited in claim 1, wherein the at least one part includes a plurality of parts spaced about the at least one opening.

7. A spinal implant as recited in claim 1, wherein the at least one part includes a plurality of inward radial projections disposed circumferentially about the at least one opening.

8. (canceled)

9. A spinal implant as recited in claim 1, wherein the fastener is removable from the wall.

10. (canceled)

11. A spinal implant as recited in claim 1, wherein the groove surface defines a perpendicular shaped groove.

12. A spinal implant as recited in claim 1, wherein the groove surface defines a V-shaped groove.

13. A spinal implant as recited in claim 1, wherein the groove surface includes at least one flat.

14. A spinal implant as recited in claim 1, wherein the groove surface includes a plurality of flats.

15. A spinal implant comprising:
a fastener; and
a plate comprising opposite top and bottom surfaces, the plate comprising an inner surface defining at least one opening that extends through the surfaces, the plate including at least one resilient part extending within the opening, the fastener comprising a groove and a ramp,
the at least one resilient part comprising a tip configured for positioning between a locked orientation in which the tip is positioned within the groove to lock the fastener with the plate and a non-locked position in which the tip is movable during engagement with the ramp, the tip being positioned below the bottom surface in the locked and non-locked orientations.

16. A spinal implant as recited in claim 15, wherein a groove surface of the fastener defines the groove, the at least one part being engageable with the groove surface when the tip is in the locked orientation.

17. A spinal implant as recited in claim 15, wherein the fastener is removable from the plate.

18-19. (canceled)

20. A spinal implant system comprising;
a bone screw; and
a wall comprising opposite to and bottom surfaces, the wall comprising an inner surface defining at least one opening that extends through the surfaces, the wall including at least one part extending within the opening,
the bone screw defining an axis and including a head and a tissue penetrating shaft, the bone screw further including a groove surface that defines a groove disposed between the head and the shaft and a ramp,
the at least one part comprising a tip configured for positioning between a locked orientation in which the tip engages the groove surface to resist and/or prevent axial movement of the bone screw relative to the wall and a non-locked position in which the tip is movable during engagement with the ramp, the tip being positioned below the bottom surface in the locked and non-locked orientations.

21. A spinal implant as recited in claim 1, wherein the fastener includes an inner top surface that faces an inner bottom surface of the fastener, the groove surface being positioned between the inner top and bottom surfaces such that the inner top and bottom surfaces and the groove surface define a groove, the tip being positioned in the groove when the tip is in the locked orientation.

22. A spinal implant as recited in claim 1, wherein the bottom surface is configured to engage tissue.

23. A spinal implant as recited in claim 1, wherein the groove surface is disposed between a head and a tissue penetrating shaft of the fastener.

24. A spinal implant as recited in claim 1, wherein:
the groove surface is disposed between a head and a tissue penetrating shaft of the fastener; and
the groove surface defines a groove comprising a first end limit and a second end limit that define bounds of the groove between the tissue penetrating shaft and the head.
